SEQUENCE LISTING (2 sequences)

SEQ ID NO: 1 - DNA, 21 bp, Artificial Sequence
  Synthetic oligonucleotide for gold NP probes.
aaaaaaaaaataaaaaaaaaa

SEQ ID NO: 2 - DNA, 21 bp, Artificial Sequence
  Synthetic oligonucleotide for gold NP probes.
ttttttttttttttttttttt

(19) United States
(12) Patent Application Publication
(10) Pub. No.: US 2010/0021894 A1
(43) Pub. Date: Jan. 28, 2010

(54) Nanoparticle-Based Colorimetric Detection Of Cysteine

(75) Inventors: Chad A. Mirkin, Wilmette, IL (US); Jae-Seung Lee, Skokie, IL (US); Min Su Han, Kyung-Buk (KR); Pirmin A. Ulmann, Evanston, IL (US)

Correspondence Address: GREGORY T. PLETTA;Nanosphere, Inc., 4088 Commerical Avenue, Northbrook, IL, 60062-1829, US

(73) Assignee: NORTHWESTERN UNIVERSITY, Evanston, IL (US)

(21) Appl. No.: 12/340,556

(22) Filed: Dec. 19, 2008

Related U.S. Application Data
(60) Provisional application No. 61/015,511, filed on Dec. 20, 2007.

(51) Int. Cl. C12Q 1/68; G01N 33/68

(52) U.S. Cl. 435/6; 436/90

(57) ABSTRACT

The invention provides methods to detect cysteine which employ oligonucleotide functionalized nanoparticles.

### CROSS-REFERENCE TO RELATED APPLICATIONS

The present application claims the benefit of the filing date of U.S. application Ser. No. 61/015,511, filed on Dec. 20, 2007, the disclosure of which is incorporated by reference herein.

### STATEMENT OF GOVERNMENTAL SUPPORT

This invention was made with government support under the Department of Defense's Defense Advanced Research Projects Agency (DARPA)/Air Force Research Labs Grant No. FA8650-06-C-7617; the Air Force Office of Scientific Research (AFOSR) Grant No. F49620-01-1-0401, and the National Institutes of Health Pioneer Award No. 5 DPI OD000285-03. The government has certain rights in this invention

### BACKGROUND

As a sulfur-containing amino acid, cysteine plays a crucial biological role in the human body by providing a modality for the intramolecular crosslinking of proteins through disulfide bonds to support their secondary structures and functions (Stryer, 1995). It is also a potential neurotoxin (Janaky et al., 1995; Puka-Sundvall et al., 1995; Wang et al., 2001), a biomarker for various medical conditions (Goodman et al., 2000; Liu et al., 2000), and a disease-associated physiological regulator (Droge et al., 1997; Perlman et al., 1940; Saravanan et al., 1996). A variety of methods for detecting cysteine, such as electrochemical voltammetry (Zen et al., 2001; Shahrokhian, 2001; Tseng et al., 2006; Zhao et al., 2003; Hignett et al., 2001) and fluorescence (Tcherkas, 2001; Pfeiffer et al, 1999), have been developed. Most of them, however, require complicated instrumentation, cumbersome laboratory procedures and throughput, which limits the scope of their practical applications. Recently, significant advances have been made in the development of chromophoric calorimetric sensors for detecting cysteine, and they have attracted attention due to their easy readout with the naked eye and potential for high throughput formats. However, they are also limited with respect to poor sensitivity (LODs≧about 1 μM), and, in certain cases, incompatibility with aqueous environments (Han et al., 2004; Shao et al., 2006; Wang et al., 2005; Rusin et al., 2003).

Gold nanoparticle (Au NP) assays are emerging as alternatives to more conventional chromogenic sensors. The Au NPs are attractive as calorimetric probes because of their intense optical properties (they are more highly colored than the best organic dyes), chemical tailorability, distance- and aggregate-size-dependent optical properties, and chemical stability (Yguerabide, 1998; Katz et al., 2004; Daniel et al., 2004; Templeton et al., 1999). In particular, oligonucleotide-functionalized gold nanoparticles (DNA-Au NPs) have been used to develop many assays for a wide variety of analytes, including proteins (Nam et al., 2003; Georganopoulou et al., 2005, Stoeva et al., 2006), oligonucleotides (Nam et al., Stoeva et al., 2006; Reynolds et al., 2000), certain metal ions (Lee et al., 2007; Liu et al., 2004; Liu et al., 2005) and other small organic molecules (Nam et al., 2005, Liu et al., 2006; Han et al., 2006a; Han et al., 2006b), based on their unique chemical and physical properties (Mirkin et al., 1996; Rosi et al., 2005; Storhoff et al., 2000). Assays for cysteine that are based upon unmodified Au NPs rely on non-selective cysteine adsorption on the surface of the NP to effect aggregation and a calorimetric change. This approach, while simple, lacks selectivity and has a relatively high LOD (≧about 7 μM) (Zhong et al., 2004; Okubo et al., 2007, Zhang et al., 2002; Sudeep et al., 2005).

### SUMMARY OF THE INVENTION

The invention provides a method to detect the presence or amount of cysteine in a sample. The method includes providing a first mixture comprising a complex comprising an agent that binds cysteine and associates with nucleotide mismatches, e.g., Hg²⁺, and a population of particles, such as gold colloid particles, or nanoparticles, including gold nanoparticles entirely composed of gold or those with an exterior gold shell. The population includes nanoparticles having at least a portion of the surface functionalized with one of a pair of single stranded oligonucleotides and nanoparticles having at least a portion of the surface functionalized with the other single stranded oligonucleotide of the pair. In one embodiment, the population includes gold nanoparticles having at least a portion of the surface functionalized with one of a pair of single stranded oligonucleotides and gold nanoparticles having at least a portion of the surface functionalized with the other single stranded oligonucleotide of the pair. The sequence of each oligonucleotide has sufficient complementarity to the other so that a double stranded duplex is capable of being formed. In one embodiment, the pair of oligonucleotides is capable of forming a double stranded duplex without any mismatches. In one embodiment, the pair of oligonucleotides is capable of forming a double stranded duplex having at least one internal (relative to the 3′ ends of the oligonucleotides) nucleotide mismatch. In another embodiment, the pair of oligonucleotides is capable of forming a double stranded duplex having a mismatch at the 3′ end of one of the oligonucleotides. In one embodiment, the mismatch is a T-T mismatch. In another embodiment, the mismatch is a A-A mismatch. In yet another embodiment, the mismatch is a T-C mismatch. In one embodiment, in the presence of Hg²⁺ the duplex is stabilized. In one embodiment, a nucleotide flanking the mismatched nucleotide in one of the oligonucleotides is not T. In another embodiment, a nucleotide flanking the mismatched nucleotide in one of the oligonucleotides is not G. In yet another embodiment, a nucleotide flanking the mismatched nucleotide in one of the oligonucleotides is not T or G. The first mixture and a sample suspected of having cysteine are mixed, forming a second mixture. Then the melting point of the double stranded duplex in the second mixture is detected or determined. The melting point of the second mixture is indicative of the presence or amount of cysteine in the sample.

In one embodiment, Hg²⁺ and a population gold nanoparticles which includes gold nanoparticles having one of a pair of single stranded oligonucleotides and gold nanoparticles having the other single stranded oligonucleotide of the pair are mixed. To that mixture is added a test sample which may contain cysteine. In one embodiment, the resulting sample is heated and the optical properties detected at various temperatures so as to identify the temperature at which the duplex denaturates. The temperature at which the duplex denatures may be compared to a standard curve to detect the amount of cysteine in the sample. Alternatively, individual samples are each heated to one temperature and the optical property of each sample is detected, e.g., for a change from purple to red.

The invention also provides a method to detect the presence of cysteine in a sample. The method includes providing a first mixture comprising complexes comprising an agent that binds cysteine and associates with nucleotide mismatches, e.g., Hg²⁺, and a population of gold nanoparticles. The population has gold nanoparticles with one of a pair of single stranded oligonucleotides and gold nanoparticles with the other single stranded oligonucleotide of the pair. The pair is selected so as to form a double stranded duplex having at least one internal nucleotide mismatch. The first mixture is contacted with a sample suspected of having cysteine to form a second mixture and then the optical properties of the second mixture are detected at one or more temperatures, e.g., a temperature selected to denature the double stranded duplex relative to a corresponding second mixture with a sample that lacks cysteine.

The invention further provides a method of detecting cysteine in sample. The method includes contacting a sample, a first nanoparticle and a second nanoparticle to form a mixture. In one embodiment, the concentration of each of the nanoparticles in the mixture is about 0.1 nM to about 10 nM. The first nanoparticle surface is functionalized on at least a portion of the surface with a first oligonucleotide and the second nanoparticle surface is functionalized on at least a portion of the surface with a second oligonucleotide. The sequence of the first oligonucleotide and the sequence of the second oligonucleotide have sufficiently complementarity to form a duplex. The mixture is subjected to conditions that provide for duplex formation and then an optical property of the mixture, for instance, at about 518 nm to about 550 nm, is detected at a temperature sufficient to denature the duplex. When the sample comprises cysteine, the optical property of the mixture is different than the optical property of the mixture in the absence of cysteine. In one embodiment, the optical property of the mixture is correlated to a melting temperature of the duplex. In one embodiment, the duplex comprises at least one mismatch, e.g., a T-T mismatch, which is at an internal nucleotide position of at least one of the oligonucleotides or at the 3′ most nucleotide position of one of the oligonucloetides. In one embodiment, at least one of the oligonucleotides has 50 nucleotides or less nucleotides. In one embodiment, at least one of oligonucleotides has at least 7 nucleotides 5′, 3′, or both 5′ and 3′ to the mismatch. In one embodiment, the contacting is carried out in the presence of mercuric ion. In one embodiment, at least one of nanoparticle types has a diameter of about 5 nm to about 200 nm, e.g., about 5 nm to about 40 nm. In one embodiment, at least one of the nanoparticle types comprises a gold nanoparticle. In one embodiment, the sample is a physiological sample from a mammal, e.g., a human, such as a plasma sample. In one embodiment, the sample is a mammalian tissue sample, such as a brain, liver, heart, or muscle tissue sample. In one embodiment, for a physiological sample of a mammal, the concentration of cysteine is correlated to the risk of one or more disorders, such as neuronal degeneration, muscle wasting or immune dysfunction.

Further provided is a method of detecting the presence or amount of cysteine a sample. The method includes contacting a sample, a first nanoparticle and a second nanoparticle to form a mixture. The first nanoparticle surface is functionalized on at least a portion of the surface with a first oligonucleotide and the second nanoparticle surface is functionalized on at least a portion of the surface with a second oligonucleotide. The sequence of the first nanoparticle and the sequence of the second nanoparticle have sufficiently complementary to form a duplex. After the mixture is subjected to conditions that provide for duplex formation, a melting temperature of the duplex in the mixture is detected. The melting temperature is indicative of the presence or amount of cysteine in the sample, when compared to a standard measurement. In one embodiment, the duplex comprises at least one mismatch, e.g., a T-T mismatch, which is at an internal nucleotide position of at least one of the oligonucleotides or at the 3′ most nucleotide position of one of the oligonucelotides. In one embodiment, at least one of the oligonucleotides has 50 nucleotides or less. In one embodiment, at least one of oligonucleotides has at least 7 nucleotides 5′, 3′ or both 5′ and 3′ to the mismatch. In one embodiment, the contacting is carried out in the presence of mercuric ion. In one embodiment, at least one of nanoparticle types has a diameter of about 5 nm to about 200 nm, e.g., about 5 nm to about 40 nm. In one embodiment, at least one of the nanoparticle types comprises a gold nanoparticle. In one embodiment, the sample is a physiological sample from a mammal, e.g., a human, such as a plasma sample. In one embodiment, the sample is a mammalian tissue sample, such as a brain, liver, heart, or muscle tissue sample. In one embodiment, for a physiological sample of a mammal, the concentration of cysteine is correlated to the risk of one or more disorders, such as neuronal degeneration, muscle wasting, and immune dysfunction.

### BRIEF DESCRIPTION OF THE FIGURES

FIG. 1. Colorimetric detection of cysteine using DNA-Au NPs in a competition assay format.

FIG. 2. A) Normalized melting transitions of DNA-Au NP/Hg²⁺ complex aggregates with different concentrations of cysteine. B) Tₘs of the melting transitions in FIG. 2A with respect to the concentration of cysteine.

FIG. 3. The colorimetric response of the DNA-Au NP/Hg²⁺ complex aggregates in the presence of the various amino acids (each at 1 μM) at 50° C.

FIG. 4. The difference of the Tₘs of the blank and the amino acid samples (each at 1 μM), and their normalized melting profiles (inset).

### DETAILED DESCRIPTION OF THE INVENTION

### Definitions

A “nucleotide” is a subunit of a nucleic acid comprising a purine or pyrimidine base group, a 5-carbon sugar and a phosphate group. The 5-carbon sugar found in RNA is ribose. In DNA, the 5-carbon sugar is 2′-deoxyribose. The term also includes analogs of such subunits, such as a methoxy group (MeO) at the 2′ position of ribose.

A “detectable moiety” is a label molecule attached to, or synthesized as part of, a polynucleotide. These detectable moieties include but are not limited to radioisotopes, calorimetric, fluorometric or chemiluminescent molecules, enzymes, haptens, redox-active electron transfer moieties such as transition metal complexes, metal labels such as silver or gold particles, or even unique oligonucleotide sequences.

A “biological sample” can be obtained from an organism, e.g., it can be a physiological fluid or tissue sample, such as one from a human patient, a laboratory mammal such as a mouse, rat, pig, monkey or other member of the primate family, by drawing a blood sample, sputum sample, spinal fluid sample, a urine sample, a rectal swab, a peri-rectal swab, a nasal swab, a throat swab, or a culture of such a sample.

“Tₘ” refers to the temperature at which 50% of the duplex is converted from the hybridized to the unhybridized form.

One skilled in the art will understand that the oligonucleotides useful in the methods can vary in sequence. The oligonucleotide pairs may have less than 100% sequence identity due to the presence of at least one mismatch. Thus, the percentage of identical bases or the percentage of perfectly complementary bases between the oligonucleotides is less than 100% but in the region of complementarity have at least 80%, 85%, 90%, 95%, 98%, or 99% identity. The oligonucleotides may also contain sequences that have no complementarity. For instance, 5′ HS-C₁₀-A₁₀T-A₁₀ 3′ (SEQ ID NO:1) and 5′ HS-C₁₀-T₁₀-T-T₁₀ 3′ (SEQ ID NO:2) have two regions of complementarity (A₁₀ and T₁₀), a mismatch flanked by the regions of complementarity and a region that is does not have complementarity (C₁₀). The oligonucleotide sequences that do not have complementarity do not prevent the pair from hybridizing.

By “sufficiently complementary” or “substantially complementary” is meant nucleic acids having a sufficient amount of contiguous complementary nucleotides to form a hybrid that is stable.

“RNA and DNA equivalents” refer to RNA and DNA molecules having the same complementary base pair hybridization properties. RNA and DNA equivalents have different sugar groups (i.e., ribose versus deoxyribose), and may differ by the presence of uracil in RNA and thymine in DNA. The difference between RNA and DNA equivalents do not contribute to differences in substantially corresponding nucleic acid sequences because the equivalents have the same degree of complementarity to a particular sequence.

As used herein, a “type of oligonucleotides” refers to a plurality of oligonucleotide molecules having the same sequence.

A “type of” nanoparticles refers to nanoparticles having the same type(s) of oligonucleotides attached to them. “Nanoparticles having oligonucleotides attached thereto” are also sometimes referred to as “nanoparticle-oligonucleotide conjugates.”

### Methods of the Invention

The invention provides sensitive methods to detect the presence or amount of cysteine in a sample. Previously, the detection of cysteine concentrations of <1 μM in chromophoric assays was not reproducible. The present methods provide for reproducible detection of cysteine levels in the range of about 100 nM to 10 μM. Further, the assays are rapid and amenable for highthroughput screening. In one embodiment, the levels of cysteine in a physiological sample, e.g., a physiological fluid sample, such as blood plasma, blood serum or saliva, or a tissue biopsy, are determined using the sensitive nanoparticle (NP)-oligonucleotide conjugate based assays of the invention.

In one embodiment, one of a pair of oligonucleotides with a mismatch is immobilized onto the surface of a population of nanoparticles and the other oligonucleotide is immobilized on a different population of nanoparticles. The oligonucleotides may be bound to the nanoparticle by any conventional means including one or more linkages between the oligonucleotides and the nanoparticle or by adsorption. In one embodiment, one or more different types of oligonucleotides are immobilized onto the surface of the nanoparticle. In one embodiment, the methods utilize a pair of oligonucleotides with a mismatch linked to gold nanoparticles complexed with Hg²⁺ to detect cysteine in an aqueous solution. The approach takes advantage of oligonucleotide hybridization events that result in the aggregation of gold nanoparticles which can significantly alter their physical properties (e.g., optical, electrical, or mechanical). The nanoparticle aggregates produced as a result of the hybridization of the pair of oligonucleotides complexed with Hg²⁺ can be disrupted by the addition of cysteine. The results of the assays described herein may allow for determining a patient at risk of or having a particular disorder that is associated with aberrant cysteine levels and/or act as a substantially more sensitive assay to measure changes in cysteine levels.

Also provided is a method of detecting cysteine in sample in which a sample, a first nanoparticle and a second nanoparticle are contacted to form a mixture. The first nanoparticle surface is functionalized on at least a portion of the surface with a first oligonucleotide and the second nanoparticle surface is functionalized on at least a portion of the surface with a second oligonucleotide. The sequence of the first oligonucleotide and the sequence of the second oligonucleotide have sufficiently complementarity to form a duplex. The mixture is subjected to conditions that provide for duplex formation and then an optical property of the mixture is detected at a temperature sufficient to denature the duplex> If the sample comprises cysteine, the optical property of the mixture is different than the optical property of the mixture in the absence of cysteine.

In another embodiment, the invention provides a method of detecting the presence or amount of cysteine a sample. The method includes contacting a sample, a first nanoparticle and a second nanoparticle to form a mixture, wherein the first nanoparticle surface is functionalized on at least a portion of the surface with a first oligonucleotide and the second nanoparticle surface is functionalized on at least a portion of the surface with a second oligonucleotide. The sequence of the first nanoparticle and the sequence of the second nanoparticle have sufficiently complementarity to form a duplex. The mixture is subjected to conditions that provide for duplex formation and a melting temperature of the duplex in the mixture is detected. The melting temperature is indicative of the presence or amount of cysteine in the sample, when compared to a standard measurement.

### Nanoparticles

In general, nanoparticles (NPs) contemplated include any compound or substance with a high loading capacity for an oligonucleotide as described herein, including for example and without limitation, a metal, a semiconductor, and an insulator particle compositions, and a dendrimer (organic or inorganic). The term “functionalized nanoparticle,” as used herein, refers to a nanoparticle having at least a portion of its surface modified with an oligonucleotide.

Thus, nanoparticles are contemplated for use in the methods which comprise a variety of inorganic materials including, but not limited to, metals, semi-conductor materials or ceramics as described in U.S. Patent Publication No 20030147966. For example, metal-based nanoparticles include those described herein. Ceramic nanoparticle materials include, but are not limited to, brushite, tricalcium phosphate, alumina, silica, and zirconia. Organic materials from which nanoparticles are produced include carbon. Nanoparticle polymers include polystyrene, silicone rubber, polycarbonate, polyurethanes, polypropylenes, polymethylmethacrylate, polyvinyl chloride, polyesters, polyethers, and polyethylene. Biodegradable, biopolymer (e.g. polypeptides such as BSA, polysaccharides, etc.), other biological materials (e.g. carbohydrates), and/or polymeric compounds are also contemplated for use in producing nanoparticles.

In one embodiment, the nanoparticle is metallic, and in various aspects, the nanoparticle is a colloidal metal. Thus, in various embodiments, nanoparticles useful in the practice of the methods include metal (including for example and without limitation, gold, silver, platinum, aluminum, palladium, copper, cobalt, indium, nickel, or any other metal amenable to nanoparticle formation), semiconductor (including for example and without limitation, CdSe, CdS, and CdS or CdSe coated with ZnS) and magnetic (for example., ferromagnetite) colloidal materials, as well as silica containing materials. Other nanoparticles useful in the practice of the invention include, also without limitation, ZnS, ZnO, Ti, TiO₂, Sn, SnO₂, Si, SiO₂, Fe, Fe⁺⁴, Ag, Cu, Ni, Al, steel, cobalt-chrome alloys, Cd, titanium alloys, AgI, AgBr, HgI₂, PbS, PbSe, ZnTe, CdTe, In₂S₃, In₂Se₃, Cd₃P₂, Cd₃As₂, InAs, and GaAs. Methods of making ZnS, ZnO, TiO₂, AgI, AgBr, HgI₂, PbS, PbSe, ZnTe, CdTe, In₂S₃, In₂Se₃, Cd₃P₂, Cd₃As₂, InAs, and GaAs nanoparticles are also known in the art. See, e.g., Weller, Angew. Chem. Int. Ed. Engl., 32, 41 (1993); Henglein, Top. Curr. Chem., 143, 113 (1988); Henglein, Chem. Rev., 89, 1861 (1989); Brus, Appl. Phys. A., 53, 465 (1991); Bahncmann, in Photochemical Conversion and Storage of Solar Energy (eds. Pelizetti and Schiavello 1991), page 251; Wang and Herron, J. Phys. Chem., 95, 525 (1991); Olshavsky, et al., J. Am. Chem. Soc., 112, 9438 (1990); Ushida et al., J. Phys. Chem., 95, 5382 (1992).

In practice, methods are provided using any suitable nanoparticle having oligonucleotides attached thereto that are in general suitable for use in detection assays known in the art to the extent and do not interfere with oligonucleotide complex formation, i.e., hybridization to form a double-strand or triple-strand complex. The size, shape and chemical composition of the particles contribute to the properties of the resulting oligonucleotide-functionalized nanoparticle. These properties include for example, optical properties, optoelectronic properties, electrochemical properties, electronic properties, stability in various solutions, magnetic properties, and pore and channel size variation. The use of mixtures of particles having different sizes, shapes and/or chemical compositions, as well as the use of nanoparticles having uniform sizes, shapes and chemical composition, is contemplated. Examples of suitable particles include, without limitation, nanoparticles, aggregate particles, isotropic (such as spherical particles) and anisotropic particles (such as non-spherical rods, tetrahedral, prisms) and core-shell particles such as the ones described in U.S. Pat. No. 7,238,472 and International Patent Publication No. WO 2002/096262, the disclosures of which are incorporated by reference in their entirety.

Methods of making metal, semiconductor and magnetic nanoparticles are well-known in the art. See, for example, Schmid, G. (ed.) Clusters and Colloids (VCH, Weinheim, 1994); Hayat, M. A. (ed.) Colloidal Gold: Principles, Methods, and Applications (Academic Press, San Diego, 1991); Massart, R., IEEE Transactions On Magnetics, 17, 1247 (1981); Ahmadi, T. S. et al., Science, 272, 1924 (1996); Henglein, A. et al., J. Phys. Chem., 99, 14129 (1995); Curtis, A. C., et al., Angew. Chem. Int. Ed. Engl., 27, 1530 (1988). Preparation of polyalkylcyanoacrylate nanoparticles prepared is described in Fattal, et al., J. Controlled Release (1998) 53: 137-143 and U.S. Pat. No. 4,489,055. Methods for making nanoparticles comprising poly(D-glucaramidoamine)s are described in Liu, et al., J. Am. Chem. Soc. (2004) 126:7422-7423. Preparation of nanoparticles comprising polymerized methylmethacrylate (MMA) is described in Tondelli, et al., Nucl. Acids Res. (1998) 26:5425-5431, and preparation of dendrimer nanoparticles is described in, for example Kukowska-Latallo, et al., Proc. Natl. Acad. Sci. USA (1996) 93:4897-4902 (Starburst polyamidoamine dendrimers).

Suitable nanoparticles are also commercially available from, for example, Ted Pella, Inc. (gold), Amersham Corporation (gold) and Nanoprobes, Inc. (gold).

Also as described in US Patent Publication No. 20030147966, nanoparticles comprising materials described herein are available commercially or they can be produced from progressive nucleation in solution (e.g., by colloid reaction), or by various physical and chemical vapor deposition processes, such as sputter deposition. See, e.g., HaVashi, (1987) Vac. Sci. Technol. July/August 1987, A5(4):1375-84; Hayashi, (1987) Physics Today, December 1987, pp. 44-60; MRS Bulletin, January 1990, pp. 16-47.

As further described in US Patent Publication No. 20030147966, nanoparticles contemplated are produced using HAuCl₄ and a citrate-reducing agent, using methods known in the art. See, e.g., Marinakos et al., (1999) Adv. Mater. 11: 34-37; Marinakos et al., (1998) Chem. Mater. 10: 1214-19; Enustun & Turkevich, (1963) J. Am. Chem. Soc. 85: 3317. Tin oxide nanoparticles having a dispersed aggregate particle size of about 140 nm are available commercially from Vacuum Metallurgical Co., Ltd. of Chiba, Japan. Other commercially available nanoparticles of various compositions and size ranges are available, for example, from Vector Laboratories, Inc. of Burlingame, Calif.

Methods of making oligonucleotides of a predetermined sequence are well-known. See, e.g., Sambrook et al., Molecular Cloning: A Laboratory Manual (2nd ed. 1989) and F. Eckstein (ed.) Oligonucleotides and Analogues, 1st Ed. (Oxford University Press, New York, 1991). Solid-phase synthesis methods are preferred for both oligoribonucleotides and oligodeoxyribonucleotides (the well-known methods of synthesizing DNA are also useful for synthesizing RNA). Oligoribonucleotides and oligodeoxyribonucleotides can also be prepared enzymatically. Non-naturally occurring nucleobases can be incorporated into the oligonucleotide, as well. See, e.g., Katz, J. Am. Chem. Soc., 74:2238 (1951); Yamane, et al., J. Am. Chem. Soc., 83:2599 (1961); Kosturko, et al., Biochemistry, 13:3949 (1974); Thomas, J. Am. Chem. Soc., 76:6032 (1954); Zhang, et al., J. Am. Chem. Soc., 127:74-75 (2005); and Zimmermann, et al., J. Am. Chem. Soc., 124:13684-13685 (2002).

At least one oligonucleotide is bound to the nanoparticle through a 5′ linkage and/or the oligonucleotide is bound to the nanoparticle through a 3′ linkage. In various aspects, at least one oligonucleotide is bound through a spacer to the nanoparticle. In these aspects, the spacer is an organic moiety, a polymer, a water-soluble polymer, a nucleic acid, a polypeptide, and/or an oligosaccharide. Methods of functionalizing the oligonucleotides to attach to a surface of a nanoparticle are well known in the art. See Whitesides, Proceedings of the Robert A. Welch Foundation 39th Conference On Chemical Research Nanophase Chemistry, Houston, Tex., pages 109-121 (1995). See also, Mucic et al. Chem. Comm. 555-557 (1996) (describes a method of attaching 3′ thiol DNA to flat gold surfaces; this method can be used to attach oligonucleotides to nanoparticles). The alkanethiol method can also be used to attach oligonucleotides to other metal, semiconductor and magnetic colloids and to the other nanoparticles listed above. Other functional groups for attaching oligonucleotides to solid surfaces include phosphorothioate groups (see, e.g., U.S. Pat. No. 5,472,881 for the binding of oligonucleotide-phosphorothioates to gold surfaces), substituted alkylsiloxanes (see, e.g. Burwell, Chemical Technology, 4:370-377 (1974) and Matteucci and Caruthers, J. Am. Chem. Soc., 103:3185-3191 (1981) for binding of oligonucleotides to silica and glass surfaces, and Grabar et al., Anal. Chem., 67:735-743 for binding of aminoalkylsiloxanes and for similar binding of mercaptoaklylsiloxanes). Oligonucleotides terminated with a 5′ thionucleoside or a 3′ thionucleoside may also be used for attaching oligonucleotides to solid surfaces. The following references describe other methods which may be employed to attach oligonucleotides to nanoparticles: Nuzzo et al., J. Am. Chem. Soc., 109:2358 (1987) (disulfides on gold); Allara and Nuzzo, Langmuir, 1:45 (1985) (carboxylic acids on aluminum); Allara and Tompkins, J. Colloid Interface Sci., 49:410-421 (1974) (carboxylic acids on copper); Iler, The Chemistry Of Silica, Chapter 6, (Wiley 1979) (carboxylic acids on silica); Timmons and Zisman, J. Phys. Chem., 69:984-990 (1965) (carboxylic acids on platinum); Soriaga and Hubbard, J. Am. Chem. Soc., 104:3937 (1982) (aromatic ring compounds on platinum); Hubbard, Acc. Chem. Res., 13:177 (1980) (sulfolanes, sulfoxides and other functionalized solvents on platinum); Hickman et al., J. Am. Chem. Soc., 111:7271 (1989) (isonitriles on platinum); Maoz and Sagiv, Langmuir, 3:1045 (1987) (silanes on silica); Maoz and Sagiv, Langmuir, 3:1034 (1987) (silanes on silica); Wasserman et al., Langmuir, 5:1074 (1989) (silanes on silica); Eltekova and Eltekov, Langmuir, 3:951 (1987) (aromatic carboxylic acids, aldehydes, alcohols and methoxy groups on titanium dioxide and silica); Lec et al., J. Phys. Chem., 92:2597 (1988) (rigid phosphates on metals).

The length of the oligonucleotide on the nanoparticle surface is typically about 15 to about 100 nucleotides. Less than 15 nucleotides may result in a oligonucleotide complex having a too low a melting temperature to be suitable in the disclosed methods. More than 100 nucleotides may result in a oligonucleotide complex having a too high melting temperature to be suitable in the disclosed methods. Thus, oligonucleotides are of about 15 to about 100 nucleotides, e.g., about 20 to about 70, about 22 to about 60, or about 25 to about 50 nucleotides in length.

Two differently functionalized nanoparticles are employed in the methods disclosed herein, each nanoparticle having a different, but at least partially complementary, oligonucleotide on its surface. Thus, the first functionalized nanoparticle comprises a first oligonucleotide on at least a portion of the surface of the first nanoparticle and the second functionalized nanoparticle comprises a second oligonucleotide on at least a portion of the surface of the second nanoparticle. The first and second oligonucleotides may be 100% complementary or may be at least about 50% complementary, but can be at least about 60%, at least about 70%, at least about 80%, or at least about 90%, 95%, 96%, 97%, 98% or 99%, but less than 100%, complementary.

### Nanoparticle Size

In various aspects, methods provided include those utilizing nanoparticles which range in size from about 1 nm to about 250 nm in mean diameter, about 1 nm to about 240 nm in mean diameter, about 1 nm to about 230 nm in mean diameter, about 1 nm to about 220 nm in mean diameter, about 1 nm to about 210 nm in mean diameter, about 1 nm to about 200 nm in mean diameter, about 1 nm to about 190 nm in mean diameter, about 1 nm to about 180 nm in mean diameter, about 1 nm to about 170 nm in mean diameter, about 1 nm to about 160 nm in mean diameter, about 1 nm to about 150 nm in mean diameter, about 1 nm to about 140 nm in mean diameter, about 1 nm to about 130 nm in mean diameter, about 1 nm to about 120 nm in mean diameter, about 1 nm to about 110 nm in mean diameter, about 1 nm to about 100 nm in mean diameter, about 1 nm to about 90 nm in mean diameter, about 1 nm to about 80 nm in mean diameter, about 1 nm to about 70 nm in mean diameter, about 1 nm to about 60 nm in mean diameter, about 1 nm to about 50 nm in mean diameter, about 1 nm to about 40 nm in mean diameter, about 1 nm to about 30 nm in mean diameter, or about 1 nm to about 20 nm in mean diameter, about 1 nm to about 10 nm in mean diameter. In other aspects, the size of the nanoparticles is from about 5 nm to about 150 nm (mean diameter), from about 5 to about 50 nm, from about 10 to about 30 nm. The size of the nanoparticles is from about 5 nm to about 150 nm (mean diameter), from about 30 to about 100 nm, from about 40 to about 80 nm. The size of the nanoparticles used in a method varies as required by their particular use or application. The variation of size is advantageously used to optimize certain physical characteristics of the nanoparticles, for example, optical properties or amount surface area that can be derivatized as described herein.

### Oligonucleotides

Each nanoparticle utilized in the methods provided has a plurality of oligonucleotides attached to it. As a result, each nanoparticle-oligonucleotide conjugate has the ability to hybridize to a second oligonucleotide functionalized on a second nanoparticle, and/or, when present, a free oligonucleotide, having a sequence sufficiently complementary. In one aspect, methods are provided wherein each nanoparticle is functionalized with identical oligonucleotides, i.e., each oligonucleotide attached to the nanoparticle has the same length and the same sequence. In other aspects, each nanoparticle is functionalized with two or more oligonucleotides which are not identical, i.e., at least one of the attached oligonucleotides differ from at least one other attached oligonucleotide in that it has a different length and/or a different sequence.

Methods of making oligonucleotides of a predetermined sequence are well-known. See, for example, Sambrook et al., Molecular Cloning: A Laboratory Manual (2nd ed. 1989) and F. Eckstein (ed.) Oligonucleotides and Analogues, 1st Ed. (Oxford University Press, New York, 1991). Solid-phase synthesis methods are contemplated for both oligoribonucleotides and oligodeoxyribonucleotides (the well-known methods of synthesizing DNA are also useful for synthesizing RNA). Oligoribonucleotides and oligodeoxyribonucleotides can also be prepared enzymatic ally.

The term “oligonucleotide” as used herein includes modified forms as discussed herein as well as those otherwise known in the art which are used to regulate gene expression. Likewise, the term “nucleotides” as used herein is interchangeable with modified forms as discussed herein and otherwise known in the art. In certain instances, the art uses the term “nucleobase” which embraces naturally-occurring nucleotides as well as modifications of nucleotides that can be polymerized. Herein, the terms “nucleotides” and “nucleobases” are used interchangeably to embrace the same scope unless otherwise noted.

In various aspects, methods include oligonucleotides which are DNA oligonucleotides, RNA oligonucleotides, or combinations of the two types. Modified forms of oligonucleotides are also contemplated which include those having at least one modified internucleotide linkage. In one embodiment, the oligonucleotide is all or in part a peptide nucleic acid. Other modified internucleoside linkages include at least one phosphorothioate linkage. Still other modified oligonucleotides include those comprising one or more universal bases. “Universal base” refers to molecules capable of substituting for binding to any one of A, C, G, T and U in nucleic acids by forming hydrogen bonds without significant structure destabilization. The oligonucleotide incorporated with the universal base analogues is able to function as a probe in hybridization, as a primer in PCR and DNA sequencing. Examples of universal bases include but are not limited to 5′-nitroindole-2′-deoxyriboside, 3-nitropyrrole, inosine and pypoxanthine.

Modified Backbones. Specific examples of oligonucleotides include those containing modified backbones or non-natural internucleoside linkages. Oligonucleotides having modified backbones include those that retain a phosphorus atom in the backbone and those that do not have a phosphorus atom in the backbone. Modified oligonucleotides that do not have a phosphorus atom in their internucleoside backbone are considered to be within the meaning of “oligonucleotide.”

Modified oligonucleotide backbones containing a phosphorus atom include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Also contemplated are oligonucleotides having inverted polarity comprising a single 3′ to 3′ linkage at the 3′-most internucleotide linkage, i.e. a single inverted nucleoside residue which may be abasic (the nucleotide is missing or has a hydroxyl group in place thereof). Salts, mixed salts and free acid forms are also contemplated. Representative United States patents that teach the preparation of the above phosphorus-containing linkages include, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, the disclosures of which are incorporated by reference herein.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages; siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts. See, for example, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, the disclosures of which are incorporated herein by reference in their entireties.

Modified Sugar and Internucleoside Linkages. In still other embodiments, oligonucleotide mimetics wherein both one or more sugar and/or one or more internucleotide linkage of the nucleotide units are replaced with “non-naturally occurring” groups. In one aspect, this embodiment contemplates a peptide nucleic acid (PNA). In PNA compounds, the sugar-backbone of an oligonucleotide is replaced with an amide containing backbone. See, for example U.S. Pat. Nos. 5,539,082; 5,714,331; and 5,719,262, and Nielsen et al., Science, 1991, 254, 1497-1500, the disclosures of which are herein incorporated by reference.

In still other embodiments, oligonucleotides are provided with phosphorothioate backbones and oligonucleosides with heteroatom backbones, and including —CH₂—NH—O—CH₂—, —CH₂—N(CH₃)—O—CH₂—, —CH₂—O—N(CH₃)—CH₂—, —CH₂ N(CH₃)—N(CH₃)—CH₂— and —O—N(CH₃)—CH₂—CH₂— described in U.S. Pat. Nos. 5,489,677, and 5,602,240. Also contemplated are oligonucleotides with morpholino backbone structures described in U.S. Pat. No. 5,034,506.

In various forms, the linkage between two successive monomers in the oligo consists of 2 to 4, desirably 3, groups/atoms selected from —CH₂—, —O—, —S—, —NR^{H}, >C═O, >C═NR^{H}, >C═S, —Si(R″)₂—, —SO—, —S(O)₂—, —P(O)₂—, —PO(BH₃)—, —P(O,S)—, —P(S)₂—, —PO(R″)—, —PO(OCH₃)—, and —PO(NHR^{H})—, where R^{H} is selected from hydrogen and C₁₋₄-alkyl, and R″ is selected from C₁₋₆-alkyl and phenyl. Illustrative examples of such linkages are —CH₂—CH₂—CH₂—, —CH₂—CO—CH₂—, —CH₂—CHOH—CH₂—, —O—CH₂—O—, —O—CH₂—CH₂—, —O—CH₂—CH═ (including R⁵ when used as a linkage to a succeeding monomer), —CH₂—CH₂—O—, —NR^{H}—CH₂—CH₂—, —CH₂—CH₂—NR^{H}—, —CH₂—NR^{H}—CH₂—, —O—CH₂—CH₂—NR^{H}—, —NR^{H}CO—O—, —NR^{H}—CO—NR^{H}—, —NR^{H}—CS—NR^{H}—, —NR^{H}—C(═NR^{H})—NR^{H}—, —NR^{H}—CO—CH₂—NR^{H}—O—CO—O—, —O—CO—CH₂—O—, —O—CH₂CO—O—, —CH₂—CO—NR^{H}—, —O—CO—NR^{H}—, —NR^{H}—CO—CH₂—, —O—CH₂—CO—NR^{H}—, —O—CH₂—CH₂—NR^{H}—, —CH═N—O—, —CH₂—NR^{H}—O—, —CH₂—O—N═ (including R⁵ when used as a linkage to a succeeding monomer), —CH₂—O—NR^{H}—, —CO—NR^{H}—CH₂—, —CH₂—NR^{H}—O—, —CH₂—NR^{H}—CO—, —O—NR^{H}—CH₂—, —O—NR^{H}, —O—CH₂—S—, —S—CH₂—O—, —CH₂—CH₂—S—, —O—CH₂—CH₂—S—, —S—CH₂—CH═ (including R⁵ when used as a linkage to a succeeding monomer), —S—CH₂—CH₂—, —S—CH₂—CH₂—O—, —S—CH₂—CH₂—S—, —CH₂—S—CH₂—, —CH₂—SO—CH₂—, —CH₂—SO₂—CH₂—, —O—SO—O—, —O—S(O)₂—O—, —O—S(O)₂—CH₂—, —O—S(O)₂—NR^{H}—, —NR^{H}—S(O)₂—CH₂—; —O—S(O)₂—CH₂—, —O—P(O)₂—O—, —O—P(O,S)—O—, —O—P(S)₂—O—, —S—P(O)₂—O—, —S—P(O,S)—O—, —S—P(S)₂—O—, —O—P(O)₂—S—, —O—P(O,S)—S—, —O—P(S)₂—S—, —S—P(O)₂—S—, —S—P(O,S)—S—, —S—P(S)₂—S—, —O—PO(R″)—O—, —O—PO(OCH₃)—O—, —O—PO(OCH₂CH₃)—O—, —O—PO(OCH₂CH₂S—R)—O—, —O—PO(BH₃)—O—, —O—PO(NHR^{N})—O—, —O—P(O)₂—NR^{H}H—, —NR^{H}—P(O)₂—O—, —O—P(O,NR^{H})—O—, —CH₂—P(O)₂—O—, —O—P(O)₂—CH₂—, and —O—Si(R″)₂—O—; among which —CH₂—CO—NR^{H}—, —CH₂—NR^{H}—, —S—CH₂—O—, —O—P(O)₂—O—O—P(—O,S)—O—, —O—P(S)₂—O—, —NR^{H}P(O)₂—O—, —O—P(O,NR^{H})—O—, —O—PO(R″)—O—, —O—PO(CH₃)—O—, and —O—PO(NHR^{N})—O—, where RH is selected form hydrogen and C₁₋₄-alkyl, and R″ is selected from C₁₋₆-alkyl and phenyl, are contemplated. Further illustrative examples are given in Mesmaeker et. al., Current Opinion in Structural Biology 1995, 5, 343-355 and Susan M. Freier and Karl-Heinz Altmann, Nucleic Acids Research, 1997, vol 25, pp 4429-4443.

Still other modified forms of oligonucleotides are described in detail in U.S. Patent Publication No. 20040219565, the disclosure of which is incorporated by reference herein in its entirety.

Modified oligonucleotides may also contain one or more substituted sugar moieties. In certain aspects, oligonucleotides comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Other embodiments include O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃]₂, where n and m are from 1 to about 10. Other oligonucleotides comprise one of the following at the 2′ position: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH2, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. In one aspect, a modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504) i.e., an alkoxyalkoxy group. Other modifications include 2′-dimethylaminooxyethoxy, i.e., a O(CH₂)₂ON(CH₃)₂ group, also known as 2′-DMAOE, as described in examples herein below, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)₂, also described in examples herein below.

Still other modifications include 2′-methoxy (2′-O—CH₃), 2′-aminopropoxy (2′-OCH₂CH₂CH₂NH₂), 2′-allyl (2′-CH₂—CH═CH₂), 2′-O-allyl (2′-O—CH₂—CH═CH₂) and 2′-fluoro (2′-F). The 2′-modification may be in the arabino (up) position or ribo (down) position. In one aspect, a 2′-arabino modification is 2′-F. Similar modifications may also be made at other positions on the oligonucleotide, for example, at the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. See, for example, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, the disclosures of which are incorporated by reference in their entireties herein.

In one aspect, a modification of the sugar includes Locked Nucleic Acids (LNAs) in which the 2′-hydroxyl group is linked to the 3′ or 4′ carbon atom of the sugar ring, thereby forming a bicyclic sugar moiety. The linkage is in certain aspects is a methylene (—CH₂—)ₙ group bridging the 2′ oxygen atom and the 4′ carbon atom wherein n is 1 or 2. LNAs and preparation thereof are described in WO 98/39352 and WO 99/14226.

Natural and Modified Bases. Oligonucleotides may also include base modifications or substitutions. As used herein, “unmodified” or “natural” bases include the purine bases adenine (A) and guanine (G), and the pyrimidine bases thymine (T), cytosine (C) and uracil (U). Modified bases include other synthetic and natural bases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil(pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-aminoadenine, 8-azaguanine and 8-azaadenine, 7-deazaguanine and 7-deazaadenine and 3-deazaguanine and 3-deazaadenine. Further modified bases include tricyclic pyrimidines such as phenoxazine cytidine(1H-pyrimido[5,4-b][1,4]benzoxazin-2(3H)-one), phenothiazine cytidine(1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzox-azin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified bases may also include those in which the purine or pyrimidine base is replaced with other heterocycles, for example 7-deaza-adenine, 7-deazaguanosine, 2-aminopyridine and 2-pyridone. Further bases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these bases are useful for increasing the binding affinity and include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. and are, in certain aspects combined with 2′-O-methoxyethyl sugar modifications. See, U.S. Pat. No. 3,687,808, U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; 5,750,692 and 5,681,941, the disclosures of which are incorporated herein by reference.

A “modified base” or other similar term refers to a composition which can pair with a natural base (e.g., adenine, guanine, cytosine, uracil, and/or thymine) and/or can pair with a non-naturally occurring base. In certain aspects, the modified base provides a Tₘ differential of 15, 12, 10, 8, 6, 4, or 2° C. or less. Exemplary modified bases are described in EP 1 072 679 and WO 97/12896.

Nanoparticles for use in the methods provided are functionalized with an oligonucleotide, or modified form thereof, which is from about 5 to about 100 nucleotides in length. Methods are also contemplated wherein the oligonucleotide is about 5 to about 90 nucleotides in length, about 5 to about 80 nucleotides in length, about 5 to about 70 nucleotides in length, about 5 to about 60 nucleotides in length, about 5 to about 50 nucleotides in length about 5 to about 45 nucleotides in length, about 5 to about 40 nucleotides in length, about 5 to about 35 nucleotides in length, about 5 to about 30 nucleotides in length, about 5 to about 25 nucleotides in length, about 5 to about 20 nucleotides in length, about 5 to about 15 nucleotides in length, about 5 to about 10 nucleotides in length, and all oligonucleotides intermediate in length of the sizes specifically disclosed to the extent that the oligonucleotide is able to achieve the desired result. Accordingly, oligonucleotides of 5, 6, 7, 8, 9, 10, 11, 12, 13, 14, 15, 16, 17, 18, 19, 20, 21, 22, 23, 24,25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, and 100 nucleotides in length are contemplated.

“Hybridization,” which is used interchangeably with the term “complex formation” herein, means an interaction between two or three strands of nucleic acids by hydrogen bonds in accordance with the rules of Watson-Crick DNA complementarity, Hoogstein binding, or other sequence-specific binding known in the art. Hybridization can be performed under different stringency conditions known in the art.

In various aspects, the methods include use of two or three oligonucleotides which are 100% complementary to each other, i.e., a perfect match, while in other aspects, the individual oligonucleotides are at least (meaning greater than or equal to) about 95% complementary to each over the all or part of length of each oligonucleotide, at least about 90%, at least about 85%, at least about 80%, at least about 75%, at least about 70%, at least about 65%, at least about 60%, at least about 55%, at least about 50%, at least about 45%, at least about 40%, at least about 35%, at least about 30%, at least about 25%, at least about 20% complementary to each other.

It is understood in the art that the sequence of the oligonucleotide used in the methods need not be 100% complementary to each other to be specifically hybridizable. Moreover, oligonucleotide may hybridize to each other over one or more segments such that intervening or adjacent segments are not involved in the hybridization event (e.g., a loop structure or hairpin structure). Percent complementarity between any given oligonucleotide can be determined routinely using BLAST programs (basic local alignment search tools) and PowerBLAST programs known in the art (Altschul et al., J. Mol. Biol., 1990, 215, 403-410; Zhang and Madden, Genome Res., 1997, 7, 649-656).

The stability of the oligonucleotide hybrids is chosen to be compatible with the assay conditions. This may be accomplished by designing the oligonucleotides in such a way that the Tₘ will be appropriate for standard conditions to be employed in the assay. In one embodiment, the nucleotide sequence is chosen so that the mismatched pair has a Tₘ in the presence of, for example, Hg²⁺, and cysteine that is different, e.g., by about 2 to 20° C., than the Tₘ of the mismatched pair in the presence of Hg²⁺ but not cysteine. The base composition of the oligonucleotides is not significant so long at the mismatched pair in the presence of an agent such as Hg²⁺ but not cysteine has greater thermal stability.

The position at which the mismatch occurs may be chosen to minimize the instability of hybrids in the presence of the cysteine binding agent, for instance, Hg²⁺. This may be accomplished by increasing the length of perfect complementarity on either side of the mismatch, as the longest stretch of perfectly homologous base sequence is ordinarily the primary determinant of hybrid stability. In one embodiment, the regions of complementarity may include G:C rich regions of homology. In one embodiment, the difference in Tₘ between samples with and without cysteine is at least 5° C. The length of the sequence may be a factor when selecting oligonucleotides for use with NPs. In one embodiment, at least one of the oligonucleotides has 50 or fewer nucleotides, e.g., has 10 to 50, 20 to 40, 15 to 30, or any integer in between 10 and 50, nucleotides. Oligonucleotides having extensive self-complementarity should be avoided.

### Exemplary Solid Substrates

Any substrate which allows observation of a detectable change, e.g., an optical change, may be employed in the methods of the invention. Suitable substrates include transparent solid surfaces (e.g., glass, quartz, plastics and other polymers), opaque solid surface (e.g., white solid surfaces, such as TLC silica plates, filter paper, glass fiber filters, cellulose nitrate membranes, nylon membranes), and conducting solid surfaces (e.g., indium-tin-oxide (ITO), silicon dioxide (SiO₂), silicon oxide (SiO), silicon nitride, etc.)). The substrate can be any shape or thickness, but generally is flat and thin. In one embodiment, the substrates are transparent substrates such as glass (e.g., glass slides) or plastics (e.g., wells of microtiter plates).

In one embodiment, the present invention relates to the detection of metallic nanoparticles on a transparent substrate. The substrate may be a multi-well plate with a plurality of wells. One of the wells on the substrate may be a test well (containing a test sample). Another one of the wells may contain a control well or a second test well. Further provided is a method for automatically detecting cysteine levels for at least some of the wells on the multi-well substrate.

### Complex Detection

Regardless of the type of oligonucleotide-binding molecule being identified, methods are provided wherein oligonucleotide complex formation (or separation) is detected by an observable change. In one aspect, complex formation (or separation) gives rise to a color change which is observed with the naked eye or spectroscopically. When using gold nanoparticles, a red-to-blue color change occurs with nanoparticle aggregation which often is detected with the naked eye. A blue-to-red color change occurs with nanoparticle de-aggregation, which is also detectable with the naked eye. In another aspect, oligonucleotide complex formation gives rise to aggregate formation which is observed by electron microscopy or by nephelometry. Aggregation of nanoparticles in general also gives rise to decreased plasmon resonance. In still another aspect, complex formation gives rise to precipitation of aggregated nanoparticles which is observed with the naked eye or microscopically.

The observation of a color change with the naked eye is, in one aspect, made against a background of a contrasting color. For instance, when gold nanoparticles are used, the observation of a color change is facilitated by spotting a sample of the hybridization solution on a solid white surface (such as, without limitation, silica or alumina TLC plates, filter paper, cellulose nitrate membranes, nylon membranes, or a C-18 silica TLC plate) and allowing the spot to dry. Initially, the spot retains the color of the hybridization solution, which ranges from pink/red, in the absence of hybridization, to purplish-red/purple, if there has been hybridization. On drying at room temperature or 80 ° C. (temperature is not critical), a blue spot develops if the nanoparticle-oligonucleotide conjugates had been linked by hybridization prior to spotting. In the absence of hybridization, the spot is pink. The blue and the pink spots are stable and do not change on subsequent cooling or heating or over time providing a convenient permanent record of the test. No other steps (such as a separation of hybridized and unhybridized nanoparticle-oligonucleotide conjugates) are necessary to observe the color change.

An alternate method for visualizing the results from practice of the methods is to spot a sample of nanoparticle probes on a glass fiber filter (e.g., Borosilicate Microfiber Filter, 0.7 micron pore size, grade FG75, for use with gold nanoparticles 13 nm in size), while drawing the liquid through the filter. Subsequent rinsing washes the excess, non-hybridized probes through the filter, leaving behind an observable spot comprising the aggregates generated by hybridization of the nanoparticle probes (retained because these aggregates are larger than the pores of the filter). This technique allows for greater sensitivity, since an excess of nanoparticle probes can be used.

Depending on experimental design, obtaining a detectable change depends on hybridization of different oligonucleotides, or disassociation of hybridized oligonucleotides, i.e., complex disassociation. Mismatches in oligonucleotide complementarity decrease the stability of the complex. It is well known in the art that a mismatch in base pairing has a much greater destabilizing effect on the binding of a short oligonucleotide probe than on the binding of a long oligonucleotide probe.

In other embodiments, the detectable change is created by labeling the oligonucleotides, the nanoparticles, or both with molecules (e.g., and without limitation, fluorescent molecules and dyes) that produce detectable changes upon hybridization of the oligonucleotides on the nanoparticles. In one aspect, oligonucleotides functionalized on nanoparticles have a fluorescent molecule attached to the terminus distal to the nanoparticle attachment terminus. Metal and semiconductor nanoparticles are known fluorescence quenchers, with the magnitude of the quenching effect depending on the distance between the nanoparticles and the fluorescent molecule. In the single-strand state, the oligonucleotides attached to the nanoparticles interact with the nanoparticles, so that significant quenching is observed. Upon polynucleotide complex formation, the fluorescent molecule will become spaced away from the nanoparticles, diminishing quenching of the fluorescence. Longer oligonucleotides give rise to larger changes in fluorescence, at least until the fluorescent groups are moved far enough away from the nanoparticle surface so that an increase in the change is no longer observed. Useful lengths of the oligonucleotides can be determined empirically. Thus, in various aspects, metallic and semiconductor nanoparticles having fluorescent-labeled oligonucleotides attached thereto are used in any of the assay formats described herein.

Methods of labeling oligonucleotides with fluorescent molecules and measuring fluorescence are well known in the art. Suitable fluorescent molecules are also well known in the art and include without limitation fluoresceins, rhodamines and Texas Red.

In yet another embodiment, two types of fluorescent-labeled oligonucleotides attached to two different particles can be used. Suitable particles include polymeric particles (such as, without limitation, polystyrene particles, polyvinyl particles, acrylate and methacrylate particles), glass particles, latex particles, Sepharose beads and others like particles well known in the art. Methods of attaching oligonucleotides to such particles are well known and routinely practiced in the art. See Chrisey et al., Nucleic Acids Research, 24, 3031-3039 (1996) (glass) and Charreyre et al., Langmuir, 13,3103-3110 (1997), Fahy et al., Nucleic Acids Research, 21,1819-1826 (1993), Elaissari et al., J. Colloid Interface Sci., 202,251-260(1998), Kolarova et al., Biotechniques, 20, 196-198 (1996) and Wolf et al., Nucleic Acids Research, 15, 2911-2926 (1987) (polymer/latex). In particular, a wide variety of functional groups are available on the particles or can be incorporated into such particles. Functional groups include carboxylic acids, aldehydes, amino groups, cyano groups, ethylene groups, hydroxyl groups, mercapto groups, and the like. Nanoparticles, including metallic and semiconductor nanoparticles, can also be used.

In aspects wherein two fluorophores are employed, the two fluorophores are designated “d” and “a” for donor and acceptor. A variety of fluorescent molecules useful in such combinations are well known in the art and are available from, e.g., Molecular Probes. An attractive combination is fluorescein as the donor and Texas Red as acceptor. The two types of nanoparticle-oligonucleotide conjugates with “d” and “a” attached are mixed, and fluorescence measured in a fluorimeter. The mixture is excited with light of the wavelength that excites d, and the mixture is monitored for fluorescence from a. Upon hybridization, “d” and “a” will be brought in proximity. In the case of non-metallic, non-semiconductor particles, hybridization is shown by a shift in fluorescence from that for “d” to that for “a” or by the appearance of fluorescence for “a” in addition to that for “d.” In the absence of hybridization, the fluorophores will be too far apart for energy transfer to be significant, and only the fluorescence of “d” will be observed. In the case of metallic and semiconductor nanoparticles, lack of hybridization will be shown by a lack of fluorescence due to “d” or “a” because of quenching as discussed herein. Hybridization is shown by an increase in fluorescence due to “a.” The person of ordinary skill in the art will readily appreciate that the discussion herein as it relates to formation of a double-strand complex, but that the use of two or three fluorophores can be utilized when a triplex polynucleotide complex is used in the method.

Other labels besides fluorescent molecules can be used, such as chemiluminescent molecules, which will give a detectable signal or a change in detectable signal upon hybridization.

Oligonucleotide complex formation (or separation) of NP aggregates, detected by any suitable means, in the presence of the (suspected) oligonucleotide-binding molecule is compared in the presence of various hairpin oligonucleotides having different sequences. Differences in the melting of complexes of the NP aggregates indicate a preference, or selectivity, of the oligonucleotide-binding molecule for the sequence of either the complex of the NP aggregates or of the hairpin oligonucleotide.

Exemplary Methods with Hg²⁺

The invention provides methods of detecting cysteine. In one embodiment, the method includes contacting a sample with a population of nanoparticles having oligonucleotides attached thereto (nanoparticle-oligonucleotide conjugates). The oligonucleotides on each nanoparticle have a sequence complementary to the sequence of an oligonucleotide on another nanoparticle as well a at least one mismatch. The contacting takes place under conditions effective to allow hybridization of the oligonucleotides on each of the types of nanoparticles in the presence of Hg²⁺. In one embodiment, nanoparticles with one of the oligonucleotides are mixed with Hg²⁺ and then nanoparticles with the other oligonucleotide are added, and the mixture is subjected to conditions allowing for hybridization. In one embodiment, nanoparticles with one of the oligonucleotides are mixed with nanoparticles with the other oligonucleotide, Hg²⁺ is added, and then the mixture is subjected to conditions allowing for hybridization. In one embodiment, nanoparticles with one of the oligonucleotides are mixed with nanoparticles with the other oligonucleotide and Hg²⁺, and the mixture is subjected to conditions allowing for hybridization. In another embodiment, nanoparticles with one of the oligonucleotides are mixed with nanoparticles with the other oligonucleotide, the mixture is subjected to conditions allowing for hybridization, and then Hg²⁺ is added. The oligonucleotides on one type of nanoparticle may all have the same sequence or may have different sequences that hybridize with different oligonucleotides, e.g., each nanoparticle may have all of the different oligonucleotides attached to it or, the different oligonucleotides may be attached to different nanoparticles.

In another embodiment, the method comprises providing a substrate having a first type of nanoparticles attached thereto. The first type of nanoparticles has oligonucleotides attached thereto, and the oligonucleotides have a sequence complementary to a first portion of the sequence of the oligonucleotide on the other type of nanoparticle. The substrate is contacted with the second type of nanoparticle under conditions effective to allow hybridization of the oligonucleotides on the nanoparticles. The oligonucleotides on one type of nanoparticles may all have the same sequence or may have different sequences that hybridize with different oligonucleotides, e.g., each nanoparticle may have all of the different oligonucleotides attached to it or the different oligonucleotides may be attached to different nanoparticles.

In one embodiment, the detectable change that occurs upon denaturation of the duplex formed between oligonucleotides on the nanoparticles may be a color change, a decrease in the amount of aggregates of the nanoparticles, or a decrease in the amount of precipitated aggregated nanoparticles. The color changes may be observed with the naked eye or spectroscopically. The aggregates of the nanoparticles may be observed by electron microscopy or by nephelometry. Precipitated aggregated nanoparticles may be observed with the naked eye or microscopically. In one embodiment, changes observable with the naked eye, e.g., a color change observable with the naked eye, are employed in the methods.

The methods of detecting cysteine based on observing a color change with the naked eye are cheap, fast, simple, robust (the reagents are stable), do not require specialized or expensive equipment, and little or no instrumentation is required. This makes them particularly suitable for use in, e.g., research and analytical laboratories in DNA sequencing, and point-of-care facilities.

The observation of a color change with the naked eye can be made more readily against a background of a contrasting color. For instance, when gold nanoparticles are used, the observation of a color change is facilitated by having a sample on a solid white surface (such as silica or alumina TLC plates, filter paper, cellulose nitrate membranes, and nylon membranes, preferably a C-18 silica TLC plate). In the case of gold nanoparticles, a pink/red color may be observed or a purple/blue color may be observed if the nanoparticles are close enough together.

While the nanoparticle complexes are based on gold nanoparticles are described in detail herein, other particles based on a wide variety of other materials (e.g., silver, platinum, mixtures of gold and silver, magnetic particles, semiconductors, quantum dots) can be used, and the particle sizes may range from 2-100 nm as described above. U.S. patent application Ser. No. 09/344,667 and PCT application WO 98/04740, both of which are incorporated herein by reference in their entirety, describe suitable nanoparticles and methods of attaching oligonucleotides to them.

### Exemplary Particle and Conjugate Preparation

Gold colloids (13 nm diameter) are prepared by reduction of HAuCl₄ with citrate as described in Frens, Nature Phys. Sci., 241:20 (1973) and Grabar, Anal. Chem., 67:735 (1995). Briefly, all glassware is cleaned in aqua regia (3 parts HCl, 1 part HNO₃), rinsed with Nanopure H₂O, then oven dried prior to use. HAuCl₄ and sodium citrate are purchased from Aldrich Chemical Company. Aqueous HAuCl₄ (1 mM, 500 mL) was brought to reflux while stirring. Then, 38.8 mM sodium citrate (50 mL) is added quickly. The solution color changes from pale yellow to burgundy, and refluxing continues for 15 minutes. After cooling to room temperature, the red solution is filtered through a Micron Separations Inc. 1 micron filter. Au colloids were characterized by UV-vis spectroscopy using a Hewlett Packard 8452A diode array spectrophotometer and by Transmission Electron Microscopy (TEM) using a Hitachi 8100 transmission electron microscope. Gold particles with diameters of 13 nm may produce a visible color change when aggregated with oligonucleotide sequences in the 10-35 nucleotide range.

Oligonucleotides may be synthesized on a 1 micromole scale using a Milligene Expedite DNA synthesizer in single column mode using phosphoramidite chemistry. Eckstein, F. (ed.) Oligonucleotides and Analogues: A Practical Approach (IRL Press, Oxford, 1991). All solutions are purchased from Milligene (DNA synthesis grade). Average coupling efficiency varies from 98 to 99.8%, and the final dimethoxytrityl (DMT) protecting group may be cleaved from the oligonucleotides to aid in purification.

For 3′-thiol-oligonucleotides, Thiol-Modifier C3 S-S CPG support is purchased from Glen Research and may be used in the automated synthesizer. During normal cleavage from the solid support (16 hours at 55° C.), 0.05 M dithiothreitol (DTT) is added to the NH₄OH solution to reduce the 3′ disulfide to the thiol. Before purification by reverse phase high pressure liquid chromatography (HPLC), excess DTT is removed by extraction with ethyl acetate.

For 5′-thiol oligonucleotides, 5′-Thiol-Modifier C₆-phosphoramidite reagent is purchased from Glen Research, 44901 Falcon Place, Sterling, Va. 20166. The oligonucleotides are synthesized, and the final DMT protecting group removed. Then, 1 ml of dry acetonitrile is added to 100 μmole of the 5′ Thiol Modifier C₆-phosphoramidite. 200 μL of the amidite solution and 200 μL of activator (fresh from synthesizer) are mixed and introduced onto the column containing the synthesized oligonucleotides still on the solid support by syringe and pumped back and forth through the column for 10 minutes. The support is then washed (2 x 1 mL) with dry acetonitrile for 30 seconds. 700 μL of a 0.016 M I₂/H₂O/pyridine mixture (oxidizer solution) is introduced into the column, and was then pumped back and forth through the column with two syringes for 30 second. The support is then washed with a 1:1 mixture of CH₃CN/pyridine (2×1 mL) for 1 minute, followed by a final wash with dry acetonitrile (2×1 mL) with subsequent drying of the column with a stream of nitrogen. The trityl protecting group is not removed, which aids in purification.

Reverse phase HPLC is performed with a Dionex DX500 system equipped with a Hewlett Packard ODS hypersil column (4.6×200 mm, 5 mm particle size) using 0.03 M Et₃NH⁺ OAc⁻ buffer (TEAA), pH 7, with a 1%/minute gradient of 95% CH₃CN/5% TEAA. The flow rate is 1 mL/minute with UV detection at 260 nm. Preparative HPLC is used to purify the DMT-protected unmodified oligonucleotides (elution at 27 minutes). After collection and evaporation of the buffer, the DMT is cleaved from the oligonucleotides by treatment with 80% acetic acid for 30 minutes at room temperature. The solution is then evaporated to near dryness, water is added, and the cleaved DMT is extracted from the aqueous oligonucleotide solution using ethyl acetate. The amount of oligonucleotide is determined by absorbance at 260 nm, and final purity assessed by reverse phase HPLC (elution time 14.5 minutes).

The same protocol is used for purification of the 3′-thiol-oligonucleotides, except that DTT is added after extraction of DMT to reduce the amount of disulfide formed. After six hours at 40° C., the DTT is extracted using ethyl acetate, and the oligonucleotides repurified by HPLC (elution time 15 minutes).

For purification of the 5′ thiol modified oligonucleotides, preparatory HPLC is performed under the same conditions as for unmodified oligonucleotides. After purification, the trityl protecting group is removed by adding 150 μL of a 50 mM AgNO₃ solution to the dry oligonucleotide sample. The sample turns a milky white color as the cleavage occurred. After 20 minutes, 200 μL of a 10 mg/mL solution of DTT is added to complex the Ag (five minute reaction time), and the sample is centrifuged to precipitate the yellow complex. The oligonucleotide solution (<50 OD) is then transferred onto a desalting NAP-5 column (Pharmacia Biotech, Uppsala, Sweden) for purification (contains DNA Grade Sephadex G-25 Medium for desalting and buffer exchange of oligonucleotides greater than 10 bases). The amount of 5′ thiol modified oligonucleotide is determined by UV-vis spectroscopy by measuring the magnitude of the absorbance at 260 nm. The final purity is assessed by performing ion-exchange HPLC with a Dionex Nucleopac PA-100 (4×250) column using a 10 mM NaOH solution (pH 12) with a 2%/minute gradient of 10 mM NaOH, 1M NaCl solution. Typically, two peaks result with elution times of approximately 19 minutes and 25 minutes (elution times are dependent on the length of the oligonucleotide strand). These peaks corresponded to the thiol and the disulfide oligonucleotides, respectively.

An aqueous solution of 17 nM (150 μL) Au colloids, prepared as described above, is mixed with 3.75 μM (46 μL) 3′-thiol-oligonucleotide, prepared as described above and allowed to stand for 24 hours at room temperature in 1 mL Eppendorf capped vials. A second solution of colloids is reacted with 3.75 μM (46 μL) 3′-thiol-complementary oligonucleotide with internal mismatch.

The oligonucleotide-modified nanoparticles are stable at elevated temperatures (80° C.) and high salt concentrations (1M NaCl) for days and do not apparently undergo particle growth. Stability in high salt concentrations is important, since such conditions are required for hybridization reactions Changes in absorbance may be recorded on a Perkin-Elmer Lambda 2 UV-vis Spectrophotometer using a Peltier PTP- 1 Temperature Controlled Cell Holder while cycling the temperature at a rate of 1° C./minute between 0° C. and 80° C. DNA solutions are approximately 1 absorbance unit(s) (OD), buffered at pH 7 using 10 mM phosphate buffer and at 1 M NaCl concentration.

There is a substantial visible optical change when the polymeric oligonucleotide-colloid precipitate is heated above its melting point. The clear solution turns dark red as the polymeric biomaterial denatures to generate the unlinked colloids which are soluble in the aqueous solution.

The following procedure is provided for attaching thiol-oligonucleotides of any length to gold colloids so that the conjugates are stable to the presence of high molecular weight DNA and hybridize satisfactorily.

A 1 mL solution of the gold colloids (17 nM) in water is mixed with excess (3.68:M) thiol-oligonucleotide (28 bases in length) in water, and the mixture is allowed to stand for 12-24 hours at room temperature. Then, 100 μL of a 0.1 M sodium hydrogen phosphate buffer, pH 7.0, and 100 μL of 1.0 M NaCl are premixed and added. After 10 minutes, 10 μL of 1% aqueous NaN₃ are added, and the mixture is allowed to stand for an additional 40 hours. This “aging” step is designed to increase the surface coverage by the thiol-oligonucleotides and to displace oligonucleotide bases from the gold surface. Somewhat cleaner, better defined red spots in subsequent assays are obtained if the solution is frozen in a dry-ice bath after the 40-hour incubation and then thawed at room temperature. Either way, the solution is next centrifuged at 14,000 rpm in an Eppendorf Centrifuge 5414 for about 15 minutes to give a very pale pink supernatant containing most of the oligonucleotide (as indicated by the absorbance at 260 nm) along with 7-10% of the colloidal gold (as indicated by the absorbance at 520 nm), and a compact, dark, gelatinous residue at the bottom of the tube. The supernatant is removed, and the residue is resuspended in about 200 μL of buffer (10 mM phosphate, 0.1 M NaCl) and recentrifuged. After removal of the supernatant solution, the residue is taken up in 1.0 mL of buffer (10 mM phosphate, 0.1 M NaCl) and 10 μL of a 1% aqueous solution of NaN₃. Dissolution is assisted by drawing the solution into, and expelling it from, a pipette several times. The resulting red master solution is stable (i.e., remained red and did not aggregate) on standing for months at room temperature, on spotting on silica thin-layer chromatography (TLC) plates, and on addition to 2 M NaCl, 10 mM MgCl₂, or solutions containing high concentrations of salmon sperm DNA.

The invention will be further described by the following non-limiting example.

### Example 1

### Materials and Methods

5′ thiol-modified oligonucleotide sequences (sequences for probe A and B, and the fluorophore-labeled DNA) were HPLC-purified and purchased from Integrated DNA Technologies (Coralville, Iowa). Au NPs (about 20 nm in diameter) were purchased from Ted Pella (Redding, Calif.). Dithiothreitol (DTT) was purchased from Pierce Biotechnology, Inc. (Rockford, Ill.). Mercury perchlorate (Hg(ClO₄)₂.xH₂O, catalog number: 529656), the twenty essential L-amino acids, and all the other chemicals were purchased from Sigma-Aldrich, and used as received.

DNA-Functionalized Au NPs were prepared following the procedure described in Lee et al. (2007). In brief, terminal disulfide groups of the DNA strands were deprotected by 0.1 M DTT in 0.17 M phosphate buffer solution (pH =8.0) for 30 minutes, purified on a NAP-5 column (GE Health Care), and added to Au NP solutions (the final oligonucleotide concentration is about 3 μM). The mixed solution was salted to 0.15 M NaCl in PBS (0.01% SDS, pH=7.4, 10 mM phosphate) and incubated overnight at room temperature. The Au NP solution was centrifuged and redispersed in 0.1 M NaNO₃, 0.005% Tween 20, 10 mM MOPS buffer (detection buffer, pH 7.5) after the supernatant was removed. The particles were washed three more times, and finally redispersed in the detection buffer. 0.7 pmol (the molar extinction coefficient of 20 nm Au NP is 8.1×10⁸ cm⁻¹M⁻¹; the molar extinction coefficient is calculated from the measured UV-Vis extinction of a colloid and a particle concentration known from the manufacturer) of each particle (probe A and B) were mixed, and incubated with Hg²⁺ ([Hg²⁺]=1 μM) overnight at 4° C. to form aggregates.

For the colorimetric detection of cysteine, a cysteine stock solution in detection buffer was mixed with the probe solution prepared as described above at room temperature to the final volume of 1 mL (the final concentration of the Au NP probes is 1.4 nM). The final concentration of cysteine ranged from 100 nM to 10 μM). Melting transition of the mixture solution was obtained shortly thereafter by monitoring the change in extinction at 528 nm as a function of temperature increased at a rate of 1° C./minute (Cary 500, Varian). The selectivity for cysteine was confirmed by adding other amino acid stock solutions to a final concentration of 1 μM instead of cysteine in a similar way.

The stability study for the Au NP probes was performed with 20 nm Au NPs functionalized with fluorophore-labeled DNA as described above. 1.4 nM of DNA-Au NPs were incubated with cysteine (0, 1, 10, and 100 μM) for one hour at room temperature or 50° C. The DNA-Au NPs were washed 2 more times with the detection buffer by centrifugation and finally redispersed in 0.5 M DTT solution in the detection buffer for 1 hour to release the fluorophore-labeled DNA from the Au NP surface. The released DNA strands were collected from the supernatant after centrifugation at 10,000 rpm for 10 minutes to precipitate the bare Au NPs. The number of DNA strands per particle was calculated from the concentration of DNA and the concentration of Au NPs.

### Results

A cysteine assay that works upon the premise of destabilization of a Au NP network connected by DNA duplexes would lead to a colorimetric assay with a sub-μM LOD, high selectivity, and quantitative output. These structures have shown promise for detecting important nucleic acid analytes with single mismatch selectivity (Elghanian et al., 1997; Storhoff et al., 1998), probing Hg²⁺ ion at nM levels (Lee et al., 2007), identifying triplex promoters (Han et al., 2006a), and screening nucleic acid (e.g., duplex DNA) intercalators (Han et al., 2006b) in a high throughput manner. As described hereinbelow, Au NP networks, interconnected with duplex DNA with strategically placed Hg²⁺-complexed thymidine-thymidine (T-T) mismatches (Miyake et al., 2006), can be used to effectively detect cysteine at a 100 nM LOD in a colorimetric format that allows one to distinguish cysteine exclusively from the 19 other essential amino acids. This assay takes advantage of the strategy of competition assays (Wiskur et al., 2001; Metzger et al., 1998; Koh et al., 1996; Han et al., 2002; Fabbrizzi et al., 2002; Hortala et al., 2002; Niikura et al., 1998; Snowden et al., 1999; Tsai et al., 2005) in combination with the sharp melting transitions and the distance-dependent optical properties of the programmable and reversible DNA-Au NP assemblies. Significantly, cysteine “competes” with the T-T mismatches for Hg²⁺, resulting in the change of the melting temperature (Tₘ) at which melting of the aggregates, or “signaling,” occurs. Unlike conventional detection methods for cysteine, the colorimetric readout can be quickly visualized with the naked eye without any spectroscopic equipment, thus making it extremely well-suited for high-throughput applications.

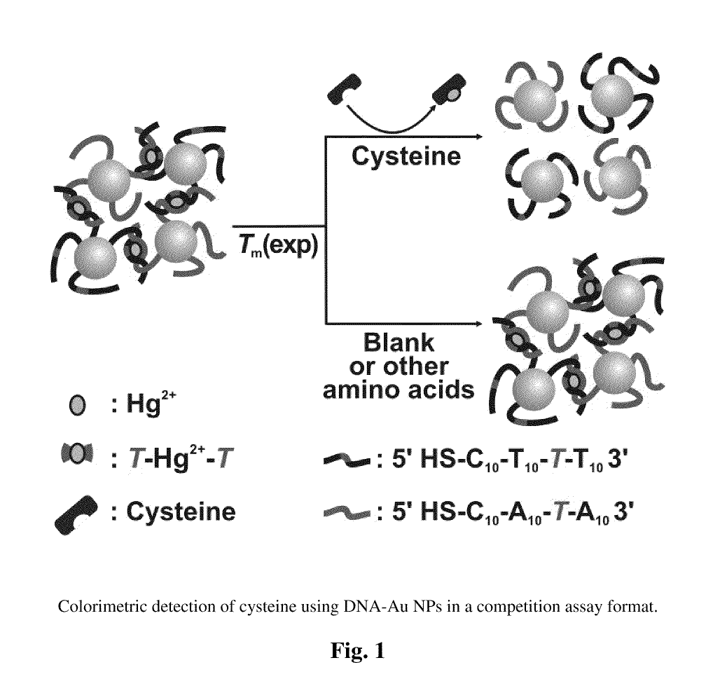

Construction of the highly sensitive and selective cysteine sensing system is shown in FIG. 1. Two sets of Au NP probes functionalized with different oligonucleotide sequences (probe A: 5′ HS-C₁₀-A₁₀T-A₁₀ 3′ (SEQ ID NO:1), probe B: 5′ HS-C₁₀-T₁₀-T-T₁₀ 3′ (SEQ ID NO:2) were prepared as described in Lee et al. (2007). When the two Au NP probes are mixed, they form aggregates through the reversible DNA-hybridization process. In general, DNA-Au NP aggregates that contain a single base mismatch dissociate at a specific temperature with a dramatic change in color and extinction (Elghanian et al., 1997; Storhoff et al., 1998). This unique melting transition also occurred when complex aggregates composed of DNA-Au NP/Hg²⁺ with T-T mismatches were heated, but at a higher temperature because of the additional stabilization induced by T-Hg²⁺-T complex formation (Lee et al., 2007). Significantly, upon the addition of cysteine, the highly thiophilic Hg²⁺ is taken out of DNA-Au NP network by the formation of Hg²⁺-cysteine complex (Cotton et al., 1999; Jalilehvand et al., 2006), thus resulting in the destabilization of DNA interconnects of DNA-Au NPs and a decrease in the Tₘ. Therefore, the concentration of cysteine is directly correlated with a decrease in the Tₘ of the DNA-Au NP/Hg²⁺ complex aggregates, providing an easy way to determine cysteine concentration.

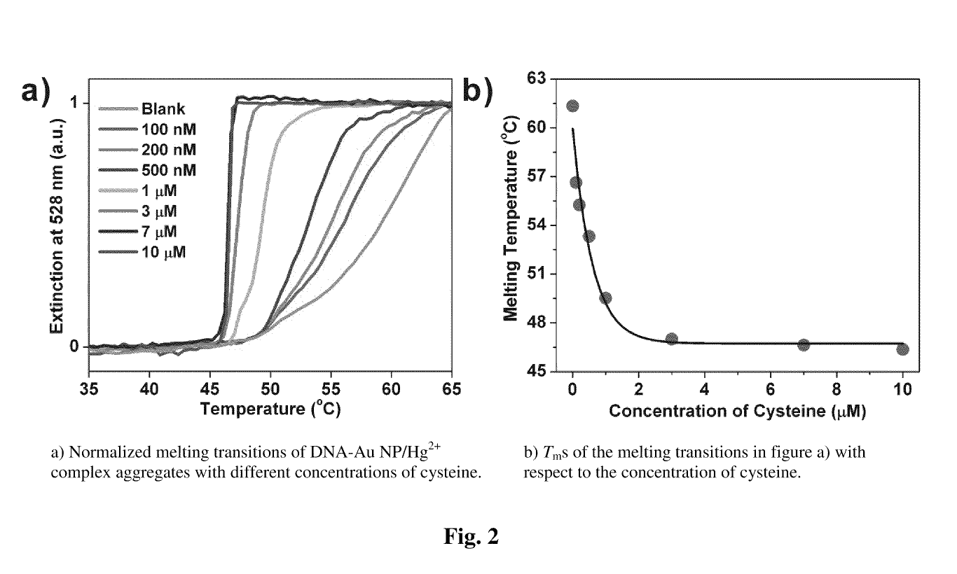

A series of concentrations of cysteine were tested to investigate the sensitivity of the assay. When a cysteine sample was mixed with the DNA-Au NP/Hg²⁺ aggregate solution, there was no detectable change in extinction at room temperature. Upon heating, however, the aggregates melted, resulting in a significant purple-to-red color change. The melting transition was obtained by heating the aggregates at a rate of 1° C./minute while monitoring the extinction at 528 nm (FIG. 2A), and the Tₘ was determined from the maximum of the first derivative of the melting transition in the visible region of the spectrum (FIG. 2B). Importantly, the observed Tₘ inversely correlates with the concentration of cysteine over the entire range of detectable cysteine concentrations studied (FIG. 2B). The limit of detection for this system is about 100 nM cysteine, which may be the lowest ever reported as a LOD distinguishable by the naked eye for a colorimetric cysteine sensing system. Each 100 nM increase in cysteine concentration results in about 0.7° C. decrease in Tₘ, and this trend is consistent up to 2 μM, allowing one to measure cysteine concentration in a quantitative way.

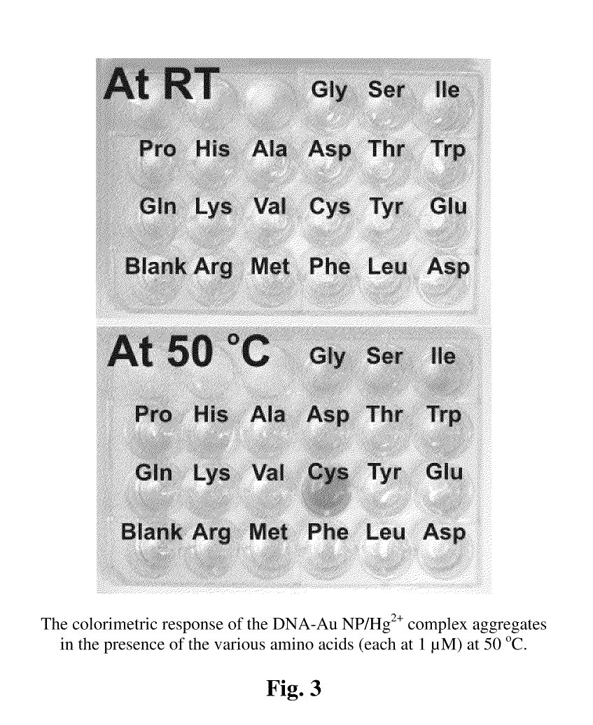

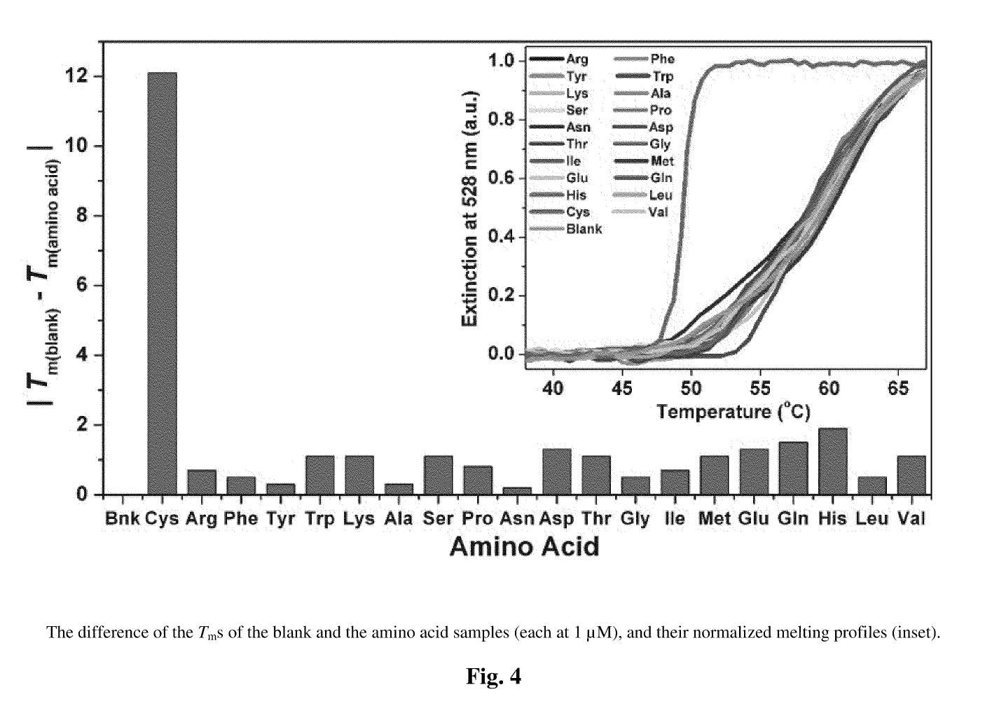

To determine the selectivity of this assay, its colorimetric response for cysteine was compared to all other 19 essential amino acids at a concentration of 1 μM (FIG. 3). A typical response to the presence of cysteine at 50° C. resulted in a dramatic color change from pale purple to dark red. In contrast to this rapid and dramatic response, the color of the aggregate solutions in the presence of all other amino acids tested remained unchanged at this temperature for the duration of the experiment (FIG. 3). The unique melting behavior of this system was further analyzed by monitoring the melting transitions (FIG. 4, inset). The melting of the aggregates without any amino acid (blank) was also monitored as a control experiment. Only the cysteine sample showed a significantly lower Tₘ (ΔTₘ=−12° C.) compared to that of the blank (FIG. 4).

This high sensitivity, selectivity, and the quantitative capabilities of the assay originate from three components: (1) the Au NPs, (2) the oligonucleotide-nanoparticle conjugate, and (3) the T-T mismatch sites in the DNA duplex. The high extinction coefficients of Au NPs (about 10⁹ cm⁻¹M⁻¹ for 20 nm Au NPs; the molar extinction coefficient is calculated from the measured UV-Vis extinction of a colloid and a particle concentration known from the manufacturer) allow nanomolar detection limits by amplifying the tiny change of the Tₘ upon binding Hg²⁺. Conventional chromogenic chemosensors have relatively low extinction coefficients (typically about 10⁵ cm⁻¹M⁻¹), which limits their sensitivity at best to the micromolar concentration range. From the standpoint of the oligonucleotide-nanoparticle conjugate, the sharp and highly cooperative melting transitions of DNA-Au NP aggregates provide a quantitative measure of the Hg²⁺ concentration over the entire concentration range studied here, from 100 nM to several micromolar concentrations, by distinguishing subtle Tₘ changes. Finally, the competition of the T-T mismatch sites with analytes for Hg²⁺ selectively excludes other amino acids besides cysteine, which has extremely high affinity for Hg²⁺ (Cotton et al., 1999). It is notable that the other sulfur-containing amino acid, methionine, did not show any significant change in Tₘ, demonstrating the preferred binding of Hg²⁺ to sulfur in a thiol group rather than sulfur in a thioether group (Sze et al., 1975). In addition, Hg²⁺ is known to have affinity for certain N-type ligands (Cotton et al., 1999), potentially including basic amino acids such as histidine or lysine. However, such a binding event between Hg²⁺ from the DNA-Au NP aggregates and basic amino acids was not observed (FIGS. 3 and 4).

It is known that thiolated molecules such as dithiothreitol remove thiolated oligonucleotides from Au surfaces (Thaxton et al., 2005). Therefore, the possibility of such displacement by cysteine through a ligand exchange process, which could result in irregular functionality of the NP probes and a loss of accuracy and sensitivity, was considered. To verify the stability of the Au NP probes toward cysteine, the number of oligonucleotides bound to the nanoparticle probe before and after conducting the assay using fluorescence spectroscopy was investigated. In this study, a thiol-modified oligonucleotide sequence labeled with a fluorophore (5′ HS-C₁₀-A₁₀T-A₁₀(6-FAM) 3′) was used to functionalize Au NPs (20 nm in diameter). The DNA-Au NPs with a fluorophore were incubated with various concentrations of cysteine (1, 10, and 100 μM over 1 hour at room temperature). The number of the DNA strands before and after the incubation was determined by measuring the fluorescence from the released DNA by dithiothreitol (Thaxton et al., 2005; Demers et al., 2000). The number of DNA strands on the Au NPs remains at almost 90% of the initial one (about 126 strands) after incubation with cysteine (Table 1). Even at higher temperature (50° C.), the displacement effect of cysteine was almost negligible (Table 1) (Dillenback et al., 2006). Concerning the increasing importance of the stability of sensing probes in various environments (Lavan et al., 2003), this demonstrated thiol-stability of DNA-Au NPs, when combined with the recent discovery of their high salt-stability (Hurst et al., 2006) in a synergetic way, provides conclusive evidence of their utility for sensing under a variety of environmentally and physiologically relevant conditions.

TABLE 1
The number of fluorophore-labeled DNA strands per
particle before and after being exposed to cysteine
at room temperature or 50° C. for 1 hour.

| | Cysteine Concentration (μM) | | | |
|---|---|---|---|---|
| | 0 | 1 | 10 | 100 |
| Room | 126.2 ± 1.3 | 121.7 ± 4.5 | 118.5 ± 5.3 | 113.0 ± 2.7 |
| Temperature | | | | |
| 50° C. | 117.3 ± 2.6 | 112.5 ± 5.4 | 106.2 ± 11.2 | 89.0 ± 2.3 |

In conclusion, a rapid, highly selective and sensitive colorimetric assay was developed for the detection of cysteine in a pool of the twenty essential amino acids using DNA-Au NPs in a competition assay format based on the high thiophilicity of Hg²⁺, and the unique optical properties and the sharp melting properties of DNA-Au NPs. In this assay, the concentration of cysteine can be determined down to 100 nM, which is more than an order of magnitude improvement over current colorimetric cysteine detection methods. The described assay is easily read by the naked eye with high accuracy, which should allow its use in point-of-care applications, e.g., to detect or determine the concentration of cysteine in a patient having a disorder associated with aberrant cysteine levels. The assay is also free from organic co-solvents, enzymatic reactions, light-sensitive dye molecules, lengthy protocols, and sophisticated instrumentation. Finally, this demonstrates, as a proof-of-concept, how one can apply a well-established strategy used in molecular systems to nanomaterial systems for detecting cysteine, which otherwise could be more cumbersome and complicated.

### References

Cotton et al., Advanced Inorganic Chemistry, 6th ed., John Wiley & Sons, Inc., New York (1999).
Daniel et al., J. Am. Chem. Soc., 104:293 (2004).
Demers et al., Anal. Chem., 72:5535 (2000).
Dillenback et al., Nano Lett., 6:16 (2006).
Droge et al., FASEB Journal, 11:1077 (1997).
Elghanian et al., Science, 277:1078 (1997).
Fabbrizzi et al., Angew. Chem. Int. Ed., 41:3811 (2002).
Georganopoulou et al., Proc. Nat. Acad. Sci., 102:2273 (2005).
Goodman et al., Cancer, 89:376 (2000).
Han et al., Angew. Chem. Int. Ed., 41:3809 (2002).
Han et al., Angew. Chem. Int. Ed., 45:1807 (2006b).
Han et al., J. Am. Chem. Soc., 128:4954 (2006a).
Han et al., Tetrahedron, 60:11251 (2004).
Hignett et al., Analyst, 126:353 (2001).
Hortala et al., J. Am. Chem. Soc., 125:20 (2002).
Hurst et al., Anal. Chem., 78:8313 (2006).
Jalilehvand et al., Inorg. Chem., 45:66 (2006).
Janaky et al., Neurochem. Res., 25:1397 (2000).
Katz et al., Angew. Chem. Int. Ed., 43:6042 (2004).
Koh et al., J. Am. Chem. Soc., 118:755 (1996).
Lavan et al., Nat. Biotechnol., 21:1184 (2003).
Lee et al., Angew. Chem. Int. Ed., epub Apr. 27, 2007.
Liu et al., Angew. Chem. Int. Ed., 45:90 (2006).
Liu et al., J. Am. Chem. Soc., 126:12298 (2004).
Liu et al., J. Am. Chem. Soc., 127:12677 (2005).
Liu et al., J. Appl. Physiol., 89:21 (2000).
Metzger et al., Angew. Chem. Int. Ed., 37:649 (1998).
Mirkin et al., Nature, 382:607 (1996).
Miyake et al., J. Am. Chem. Soc., 128:2172 (2006).
Nam et al., Anal. Chem., 77:6985 (2005).
Nam et al., J. Am. Chem. Soc., 126:5932 (2004).
Nam et al., Science, 301:1884 (2003).
Niikura et al., J. Am. Chem. Soc., 120:8533 (1998).
Okubo et al., Anal. Sci., 23:85 (2007).
Perlman et al., J. Biol. Chem., 133:651(1940).
Pfeiffer et al,. Clin. Chem., 45:290 (1999).
Puka-Sundvall et al., Brain Res., 705:65 (1995).
Reynolds et al., J. Am. Chem. Soc., 122:3795 (2000).
Rosi et al., Chem. Rev., 105:1547 (2005).
Rusin et al., J. Am. Chem. Soc., 126:438 (2003).
Saravanan et al., Br. J. Urol., 78:22 (1996).
Shahrokhian, Anal. Chem., 73:5972 (2001).
Shao et al., Angew. Chem. Int. Ed., 45:4944 (2006).
Snowden et al., Curr. Opin. Chem. Biol., 3:740 (1999).
Stoeva et al., Angew. Chem. Int. Ed., 118:3381 (2006).
Stoeva et al., J. Am. Chem. Soc., 128:8378 (2006).
Storhoff et al., J. Am. Chem. Soc., 120:1959 (1998).
Storhoff et al., J. Am. Chem. Soc., 122:4640 (2000).
Stryer, Biochemistry, 4th Ed. ed., W.H. Freeman & Company (1995).
Sudeep et al., J. Am. Chem. Soc., 127:6516 (2005).
Sze et al., Inorg. Chem., 14:1969 (1975).
Tcherkas et al., J. Chromatogr. A, 913:309 (2001).
Templeton et al., J. Am. Chem. Soc., 121:7081 (1999).
Thaxton et al., Anal. Chem., 77:8174 (2005).
Tsai et al., Chem. Commun.,4273 (2005).
Tseng et al., Electroanalysis, 18:1306 (2006).
Wang et al., J. Am. Chem. Soc., 127:15949 (2005).
Wang et al., J. Neurosci., 21:3322 (2001).
Wiskur et al., Acc. Chem. Res., 34:963 (2001).
Yguerabide et al., Anal. Biochem., 262:137 (1998).
Zen et al., Anal. Chem., 73:1169 (2001).
Zhang et al., Analyst, 127:462 (2002).
Zhao, Sens. Actuat. B, 92:279 (2003).
Zhong et al., J. Phys. Chem. B, 108:4046 (2004).

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

What is claimed is:

1. A method to detect the presence of cysteine in a sample, comprising:
a) providing a first mixture comprising complexes comprising Hg²⁺ and a population of gold nanoparticles, wherein the population comprises gold nanoparticles comprising one of a pair of single stranded oligonucleotides and gold nanoparticles comprising the other single stranded oligonucleotide of the pair, wherein the pair forms a double stranded duplex having at least one nucleotide mismatch;
b) contacting the first mixture with a sample suspected of having cysteine to form a second mixture; and
c) detecting an optical property of the second mixture at a temperature selected to denature the double stranded duplex relative to a corresponding second mixture that lacks cysteine, wherein a change in the optical property in the second mixture with the sample is associated with the presence of cysteine in the sample.

2. A method to detect the presence or amount of cysteine in a sample, comprising:
a) providing a first mixture comprising a complex comprising Hg²⁺ and a population gold nanoparticles, wherein the population comprises gold nanoparticles comprising one of a pair of single stranded oligonucleotides and gold nanoparticles comprising the other single stranded oligonucleotide of the pair, wherein the pair forms a double stranded duplex having at least one internal nucleotide mismatch;
b) contacting the first mixture with a sample suspected of having cysteine to form a second mixture; and
c) detecting the melting point of the double stranded duplex in the second mixture, wherein the melting point is indicative of the presence or amount of cysteine in the sample.

3. The method of claim 1 or 2 wherein the mismatch is a T-T mismatch.

4. The method of claim 1 or 2 wherein at least one of the pair of oligonucleotides is 50 nucleotides or less in length.

5. The method of claim 1 or 2 wherein one of the oligonucleotides has at least 7 nucleotides 5′ or 3′, or both, to the mismatch.

6. The method of claim 1 or 2 wherein the nanoparticles are about 5 to about 200 nm in diameter.

7. The method of claim 1 or 2 which detects cysteine concentrations from about 100 nM to about 10 μM.

8. The method of claim 1 wherein the optical properties are detected over a range of temperatures including the selected temperature.

9. The method of claim 1 or 2 wherein the sample is a physiological sample of a mammal.

10. The method of claim 9 wherein the sample is a plasma sample.

11. The method of claim 9 wherein the sample is from a female at risk of cervical displasia.

12. The method of claim 9 wherein the sample is a mammalian tissue sample.

13. The method of claim 12 wherein the sample is a brain, liver, heart or muscle sample.

14. The method of claim 2 wherein the melting point is correlated to the amount of cysteine in the sample.

15. The method of claim 14 wherein the sample is a physiological sample of a mammal and the amount of cysteine in the sample is correlated to the risk of neuronal degeneration.

16. The method of claim 14 wherein the sample is a physiological sample of a mammal and the amount of cysteine in the sample is correlated to the risk of muscle wasting in the mammal.

17. The method of claim 14 wherein the sample is a physiological sample of a mammal and the amount of cysteine in the sample is correlated to the risk of immune dysfunction in the mammal.

18. The method of claim 1 or 2 wherein the concentration of the population of gold nanoparticles in the first mixture is about 0.1 to about 10 nM.

19. The method of claim 1 wherein the optical property at about 518 to about 550 nm is detected.

20. The method of claim 2 wherein a sample with cysteine has a melting point at least 5° lower than a sample without cysteine.

21. A method of detecting cysteine in sample comprising
a) contacting a sample, a first nanoparticle and a second nanoparticle to form a mixture, wherein the first nanoparticle surface is functionalized on at least a portion of the surface with a first oligonucleotide and the second nanoparticle surface is functionalized on at least a portion of the surface with a second oligonucleotide, wherein the sequence of the first oligonucleotide and the sequence of the second oligonucleotide have sufficiently complementary to form a duplex, and wherein the mixture is subjected to conditions that provide for duplex formation; and
b) detecting an optical property of the mixture at a temperature sufficient to denature the duplex, wherein, when the sample comprises cysteine, the optical property of the mixture is different than the optical property of the mixture in the absence of cysteine.

22. The method of claim 21 wherein the optical property of the mixture is correlated to a melting temperature of the duplex.

23. The method of claim 21 wherein the duplex comprises at least one mismatch.

24. The method of claim 21 wherein the contacting is carried out in the presence of mercuric ion.

25. The method of claim 21 wherein the cysteine is present in the sample at a concentration of about 100 nM or greater.

26. The method of claim 22 wherein the difference between the melting temperature of duplex in the presence of cysteine compared to the melting temperature of the duplex in the absence of cysteine is 5° C. or more.

27. The method of claim 26 wherein the difference in melting temperature is 7° C. or more.

28. The method of claim 22 further comprising calculating a concentration of cysteine in the sample by comparing the melting temperature of the duplex to a standard curve comprising melting temperatures of duplexes in the presence of known concentrations of cysteine.

29. The method of claim 21 wherein the optical property comprises a color change of the mixture when the duplex denatures.

30. The method of claim 29 wherein the color change comprises a change from purple before the duplex denatures to red after the duplex denatures.
